(19) United States
(12) Patent Application Publication
(10) Pub. No.: US 2009/0043203 A1
(43) Pub. Date: Feb. 12, 2009

(54) POWER MANAGEMENT IN PORTABLE ULTRASOUND DEVICES

(76) Inventors: Laurent PELISSIER, Vancouver (CA); Kris DICKIE, Chilliwack (CA); Kwun-Keat CHAN, Vancouver (CA)

Correspondence Address: OYEN, WIGGS, GREEN & MUTALA LLP;480 - THE STATION, 601 WEST CORDOVA STREET, VANCOUVER, BC, V6B 1G1, CA

(21) Appl. No.: 12/188,186

(22) Filed: Aug. 7, 2008

Related U.S. Application Data
(60) Provisional application No. 60/955,329, filed on Aug. 10, 2007.

(51) Int. Cl. A61B 8/14

(52) U.S. Cl. 600/446

(57) ABSTRACT

A method for operating an ultrasound device comprises automatically switching among power modes in responses to changes in the power remaining available for operation of the ultrasound device. Two or more power modes may be available for each of a number of operational modes. The power modes may trade off performance against operating time. In some embodiments the ultrasound device can operate in a reduced-power idle mode in which the ultrasound device checks for ultrasound echoes indicating that a transducer is against a subject. In some embodiments, switching among power modes involves changes such as: changing a line density of ultrasound images; changing numbers of transducer elements being used for ultrasound transmission and/or reception; reconfiguring data processing circuitry; and changing pulse characteristics of transmitted ultrasound.

### CROSS-REFERENCE TO RELATED APPLICATION

This application claims the benefit under 35 USC §119 of U.S. patent application No. 60/955329 filed on 10 Aug. 2007 and entitled POWER MANAGEMENT IN PORTABLE ULTRASOUND DEVICES which is hereby incorporated by reference herein.

### TECHNICAL FIELD

This invention relates to ultrasonic devices, in particular to medical ultrasound devices. The invention has particular application to ultrasonic devices powered by batteries or other limited supplies of electrical power.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic view of a portable ultrasound device.

FIG. 2 is a block diagram showing major functional components of an example ultrasound device.

FIG. 3 is a partial schematic view of a processor system for an ultrasound device.

FIG. 4 shows an example of the contents of a memory in an ultrasound device.

FIGS. 5 illustrates a procedure for making an example ultrasound device ready to operate.

FIG. 6 illustrates a procedure for controlling power consumption in an ultrasound device.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The features as described herein may be combined in any suitable combinations with the features described in the commonly-owned US provisional patent applications entitled:
HAND-HELD ULTRASOUND SYSTEM HAVING STERILE ENCLOSURE (application No. 60/955327);
HAND-HELD ULTRASOUND IMAGING DEVICE HAVING RECONFIGURABLE USER INTERFACE (application No. 60/955328);
HAND-HELD ULTRASOUND IMAGING DEVICE HAVING REMOVABLE TRANSDUCER ARRAYS (application No. 60/955325);
WIRELESS NETWORK HAVING PORTABLE ULTRASOUND DEVICES (application No. 60/955331);
HANDHELD ULTRASOUND IMAGING SYSTEMS (application No. 60/977353) all of which are hereby incorporated herein by reference. The features as described herein may also be combined in any suitable combinations with the features described in the commonly-owned US non-provisional patent applications which are filed on the same day as the instant application and entitled:
HAND-HELD ULTRASOUND SYSTEM HAVING STERILE ENCLOSURE (claiming priority from application No. 60/955327);
HAND-HELD ULTRASOUND IMAGING DEVICE HAVING RECONFIGURABLE USER INTERFACE (claiming priority from application No. 60/955328);
HAND-HELD ULTRASOUND IMAGING DEVICE HAVING REMOVABLE TRANSDUCER ARRAYS (claiming priority from application No. 60/955325);
WIRELESS NETWORK HAVING PORTABLE ULTRASOUND DEVICES (claiming priority from application No. 60/955331); and
HANDHELD ULTRASOUND IMAGING SYSTEMS (claiming priority from application No. 60/977353)
   all of which are hereby incorporated herein by reference.

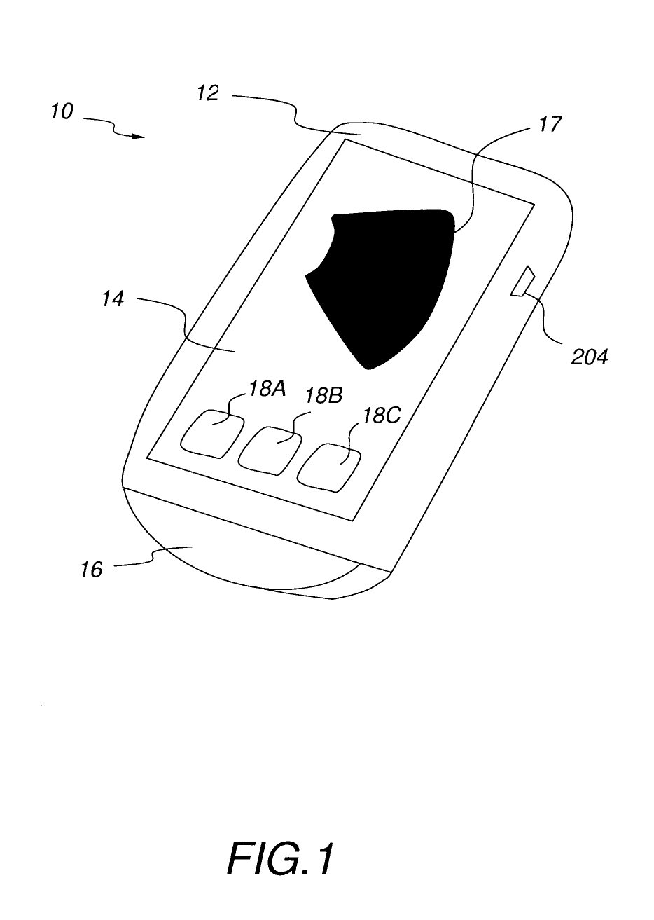

FIG. 1 shows a schematic view of a portable ultrasound device 10. Ultrasound device 10 has a housing 12 supporting a display 14 and a transducer assembly 16. A compact portable power supply within housing 12 supplies electrical power for the operation of device 10. Device 10 comprises appropriate circuits to drive transducer elements of transducer assembly 16 to emit ultrasound. The ultrasound reflects off of structures within a subject's body. Reflected ultrasound is received at transducer 16 and processed by appropriate circuits within device 10. In the illustrated embodiment, the circuits process reflected signals to generate an ultrasound image 16 which is displayed on display 14.

It is not mandatory that device 10 have a display that is displaying or that is capable of displaying an ultrasound image. In some embodiments, reflected ultrasound signals are applied to generate a result other than an image or are applied to generate image data that is saved and/or transmitted for display on a device other than device 10.

Device 10 comprises user controls that permit a user to control aspects of the operation of device 10. In the illustrated embodiment, controls 18A, 18B and 18C are defined by touch sensitive areas on display 14. Any suitable controls may be provided.

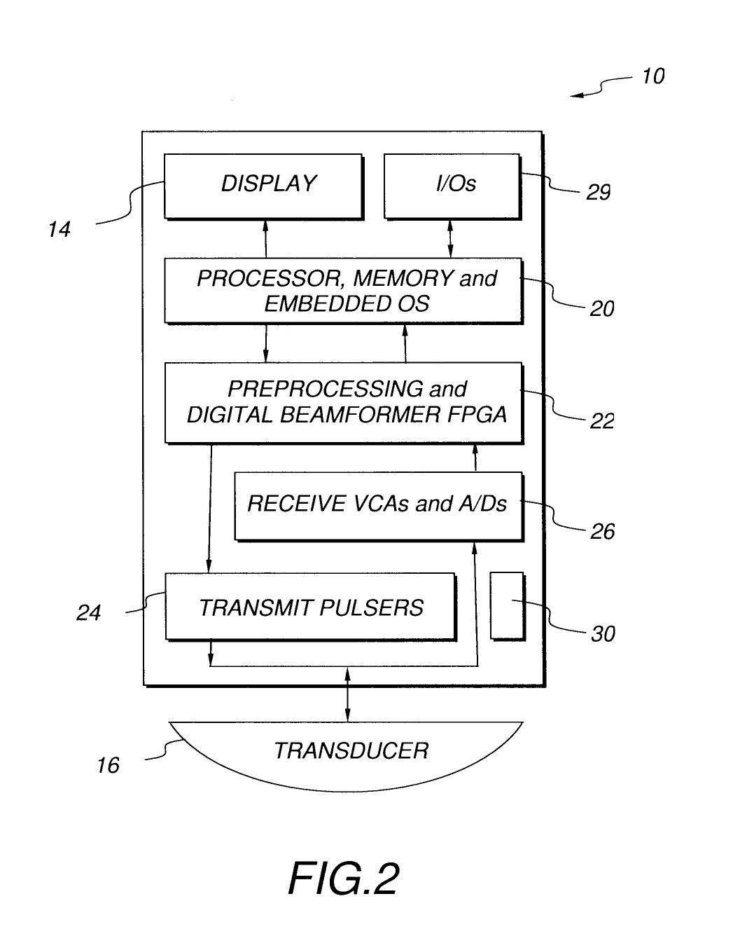

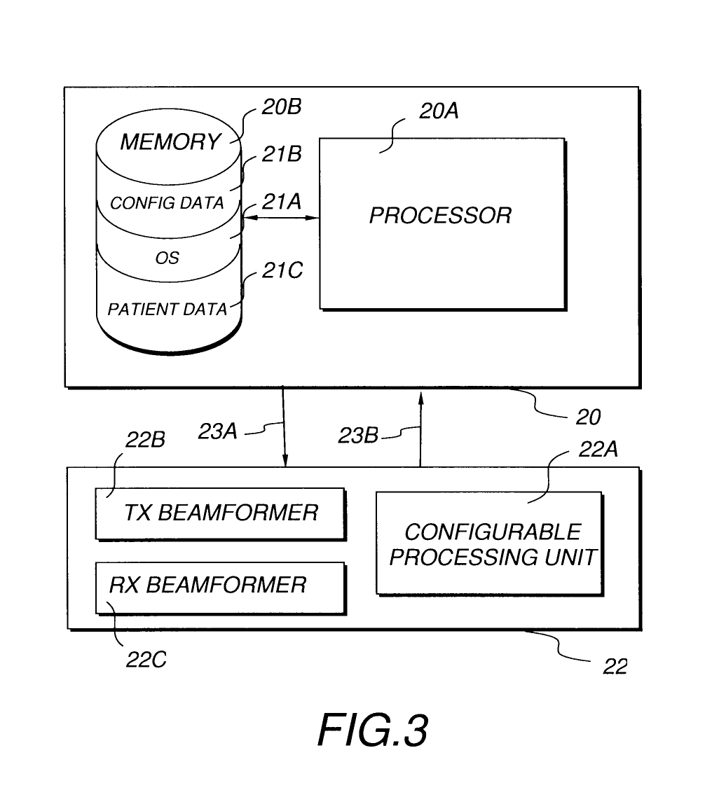

FIG. 2 shows schematically major functional components of an example embodiment of device 10. In the embodiment of FIG. 2, the operation of device 10 is coordinated by a processor system 20. Processor system 20 incorporates a program memory 20B and a data processor 20A (see FIG. 3). An operating system causes processor 20A to execute computer software instructions stored in the memory to coordinate the operation of device 10.

Signal processing is performed by a signal-processing subsystem 22 which, in the illustrated embodiment, is configurable to perform signal processing in different ways. Signal-processing subsystem 22 may, for example, comprise a Field Programmable Gate Array (FPGA) that is reconfigurable to change the way in which signals are processed. The configuration of signal-processing subsystem 22 may be controlled by processor 20.

For a particular type of diagnostic procedure or imaging, processor system 20 may retrieve a configuration file for the FPGA (or other reconfigurable parts of signal-processing subsystem 22) from its memory and send instructions to the FPGA (or other reconfigurable parts of signal-processing subsystem 22) which cause the FPGA to be configured according to the configuration file.

Under the control of processor 20 by way of signal-processing subsystem 22, instructions are given to transmit pulsing circuits 24 to deliver suitable signals to transducer assembly 16 to cause the transducer assembly 16 to emit ultrasound. Different signals may be delivered to different transducer elements of transducer assembly 16 to cause the emitted ultrasound to have desired characteristics. For example, the emission of ultrasound by different transducer elements may be timed to yield one or more directed ultrasound beams.

Reflected ultrasound pulses are received at transducer assembly 16 which generates electrical signals which are processed by receiving circuits 26 which may, for example, comprise voltage-controlled amplifiers, suitable filters or other signal conditioning circuitry, and analog-to-digital converters. Digitized reflection signals are provided to signal-processing subsystem 22 which performs at least initial processing on those signals. The resulting processed signals are then provided to processor system 20 which may be configured to display an ultrasound image 17 (see FIG. 1) on display 14 in response thereto. Signal-processing may be split between processor system 20 and signal-processing subsystem 22 in any suitable manner.

Device 10 may also have other input/output interfaces 29. By way of nonlimiting example, interfaces 29 may comprise wireless interfaces such as infrared, ultra wide bandwidth (UWB) or other wireless communication signal interfaces, and/or serial or parallel interfaces such as USB, IEEE 1394, or the like. A battery 30 or other portable power supply provides electrical power for the operation of device 10.

Device 10 may be used in surgical procedures or other situations in which the ongoing availability of device 10 is being relied upon by medical personnel (and the subject on which device 10 is being used). As such, sudden cessation of operation of the device due to exhaustion of battery 30 (or other limited power supply that might be provided in place of battery 30) could cause problems.

Device 10 has power management features which extend the operating time of device 10.

Device 10 may have power management features of the general type found in various types of electronic devices such as personal computers and the like. In addition to these features device 10 may include specific power management features that affect its operation as an ultrasound device.

General power management features that may be provided in device 10 include features such as:
Automatic dimming of a back light which illuminates display 14 as available battery power falls below a threshold and/or when device 10 is not being used.
Reduction of the clock frequency of processor 20 when device 10 is idle.
Shutting down other circuits or components such as hard drives of device 10 when device 10 is idle.

Device 10 includes a number of power management features that are specific to its functioning as a ultrasound device. These power saving features may include one or more of:
Shutting off power to ultrasound transmission and/or reception circuitry when device 10 is idle.
Reducing a frame rate of ultrasound imaging as available battery power decreases. This reduction may be made in multiple steps in some embodiments.
Reducing the line density of ultrasound imaging as battery power decreases. This reduction may be made in multiple steps.
Reducing the pulse length of ultrasound pulses transmitted by way of transducer assembly 16.
Reducing the power of ultrasound signals transmitted by way of transducer assembly 16.
Reducing the amount of processing done on data acquired from transducer assembly 16 for all frames or, in the alternative, for selected frames. Where the amount of processing is reduced for only selected frames, still images may be displayed using frames for which more processing is done.
Processing only selected frames for display (e.g., processing only every Nth frame).
Reducing the number of elements used in transducer assembly 16 for transmitting ultrasound signals.
Reducing the number of elements of an array of transducers and transducer assembly 16 used for receiving reflected ultrasound signals.

Device 10 may have a number of power modes. Different power modes may be selected based upon available battery power. In such embodiments, a first power mode may be automatically selected when the battery is fully charged. When the battery level reaches a first threshold, a lower power mode may be selected. When the battery power reaches a second, still lower threshold, a further mode which uses less power still may be selected. Device 10 may have two or more power modes.

In some embodiments, device 10 has a plurality of different operational modes. For example, device 10 may be configurable to perform a variety of different imaging tasks. The tasks may differ from one another in terms of the nature of the ultrasound signals transmitted from transducer assembly 16, the way in which signals received at transducer assembly 16 are processed and/or the way in which the processed signals are rendered into an image for display on display 14 or storage or display on some on other display. For example, one operational mode may perform B-mode imaging, another operational mode may perform Doppler processing of received signals, and so on.

Where device 10 has a plurality of different operational modes, device 10 may have a plurality of different power modes defined for each of a plurality of different operational modes—i.e. multiple power modes for each operational mode. This permits the power modes to be tailored to the requirements of the particular operational mode. For example, for an operational mode intended for use in a procedure which requires a higher frame rate, any lower power operational modes may be defined so that they maintain a relatively high frame rate but focus power saving on other aspects such as the number of transmit channels used to transit ultrasound signals, the brightness of display 14, the power of transmitted ultrasound signals, or the like.

Where signal-processing subsystem 22 includes an FPGA or other configurable signal processing circuitry, changing between power modes may involve reconfiguring the signal processing path in significant ways. For example, a high-power mode may involve separate processing of a relatively large number of channels of data from transducer assembly 16. A lower-power processing mode may involve processing data from fewer transducer elements of transducer assembly 16. In switching between the modes, the organization of a part of a signal path passing through an FPGA or other configurable electronics may be changed in a way which results in significant power saving in the FPGA. Similarly, amplifiers, filters and other signal conditioning elements for signal lines that are not used in the lower power mode may be turned off or operated at a minimal level to conserve electrical power.

FIG. 3 is a partial schematic view of a processor system 20 comprising a processor 20A and a memory 20B connected to a signal-processing subsystem 22 comprising a configurable processing unit 22A, a transmit beamformer 22B and a receive beamformer 22C. Although configurable processing unit 22A, transmit beamformer 22B and receive beam former 22C are illustrated as being separate elements in FIG. 3, transmit beamformer 22B and/or receive beamformer 22C may be provided in whole or in part by signal processing paths set up in configurable processing unit 22A.

Also, as shown in FIG. 3, memory 20B may contain an operating system 21A, configuration data 21B and patient data 21C. Configuration data 21B includes data specifying the configuration of configurable processing unit 22A for various operational and/or power consumption modes. Processor 20A executing instructions of operating system 21A can download specific configuration data 21B to configurable processing unit 22A by means of signal path 23A. Signals for controlling the operation of signal-processing subsystem 22 during operation of device 10 may also be provided by way of downstream signal path 23A. Status information regarding signal-processing subsystem 22 and processed or partially-processed data may be delivered to processor 20A from signal-processing subsystem 22 by way of upstream signal path 23B.

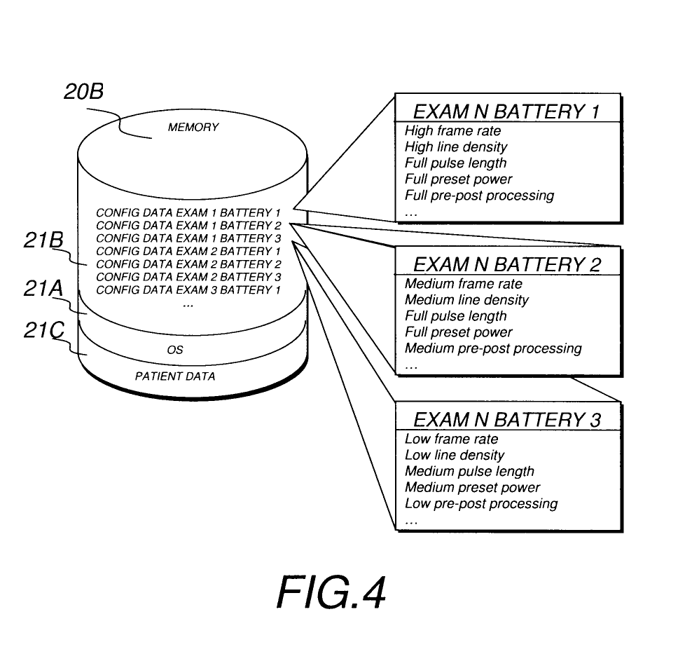

FIG. 4 shows an example of the contents of memory 20B. In the illustrated embodiment, configuration data 21B includes configuration data for a plurality of different operational modes (identified as Exam 1, Exam 2, Exam 3, . . . etc.). In the illustrated embodiment, for each operational mode there are provided three different power modes. Each power mode may specify a plurality of different parameters that affect the operation of ultrasound device 10. In particular, the different power modes affect the rate of power consumption of ultrasound device 10, while still permitting operation in the selected operational mode.

In some embodiments, the different power modes each have a specified battery level at which they are invoked automatically and these battery levels may be consistent across all operational modes. For example, when a battery has a level within 70-100%, device 10 may operate in the current operational mode in the power mode ‘battery 1’ specified for the current operational mode. If the battery level falls so that it has a value within the range of 40-70% of its capacity, then the device 10 may automatically switch to operate in a power mode of ‘battery 2’ in the current operational mode. When the battery level falls to have a value in the range of 0-40%, then the device may operate in the power mode ‘battery 3’ in the current operational mode at least until the battery no longer contains sufficient power to maintain operation of device 10.

In some embodiments, power modes may be manually selected by a user. For example, a user may invoke a particular operational mode, and may select a power mode such that the device 10 can be expected to operate for at least a specified period of time on the available battery charge. For example, a user interface may have a slider or other suitable control that the user can operate. The user may slide or operate the control in one direction to achieve high imaging performance at the expense of operating time or, may slide the user control in another direction to achieve longer battery life at the expense of imaging performance. A display on device 10 may display an indication of the estimated amount of time remaining in the current operating mode at the current power mode before battery 30 is exhausted and can no longer maintain operation of device 10, at least in the current operating mode and power mode.

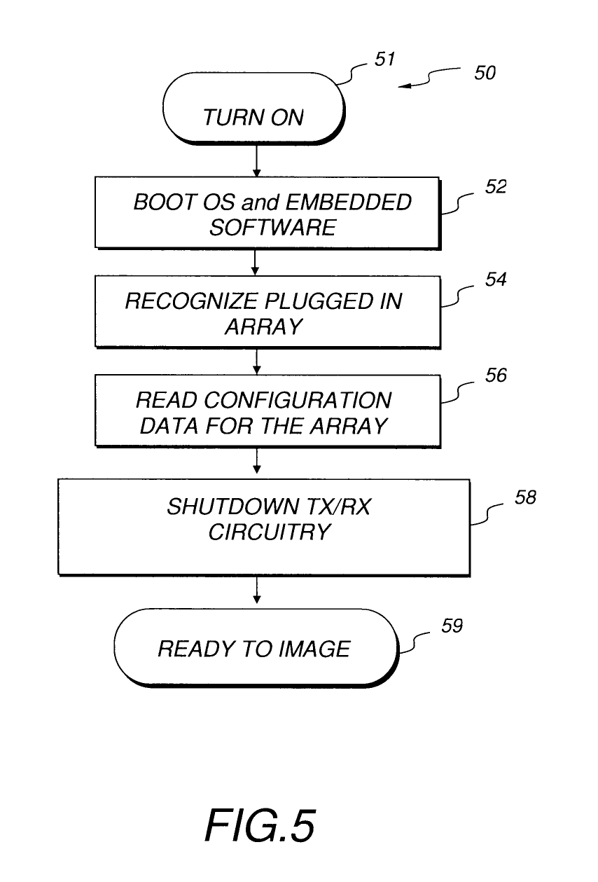

FIG. 5 illustrates a procedure for making an embodiment of device 10 ready to operate. In block 51, device 10 is turned on. In response to being turned on, in block 52, processor 20A boots using operating system 21A and invokes embedded software which controls the overall functioning of device 10. In the illustrated embodiment, the operating mode of device 10 is determined in whole or part based upon the particular transducer assembly 16 which is connected to device 10. Device 10 may be compatible with a plurality of different transducer assemblies 16, each appropriate for one or more specific operating modes. In block 54, device 10 may recognize the connected transducer assembly 16 by reading electrical signals from transducer assembly 16, or detecting a state of a switch or other device which is operated when the particular transducer assembly 16 is connected to device 10.

In block 54, the software executing in device 10 recognizes the connected transducer assembly 16. In block 56, processor 20A reads configuration data 21B for the transducer assembly 16. In block 58, transmit and receive circuitry is shutdown pending initiation of imaging. In block 59, device 10 is ready to commence imaging. When imaging commences, the transmit and receive circuitry are made operational to transmit and receive ultrasound signals.

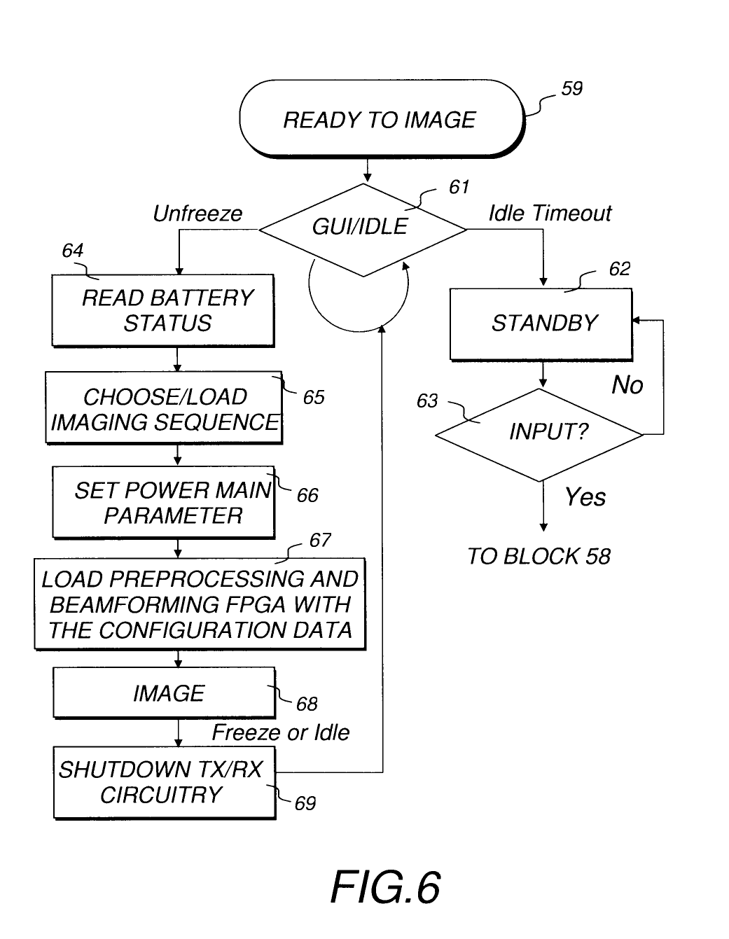

As shown in FIG. 6, when block 59 has been completed, device 10 is ready to image but is in a low power idle mode. The idle mode is schematically illustrated in FIG. 6 as an idle loop 61. In the idle mode, device 10 waits for an instruction that will cause it to commence imaging. In the idle mode, a user interface is active to detect commands from a user but device 10 is otherwise consuming little electrical power.

In some embodiments, device 10 switches from its idle mode and begins imaging when it is woken up by a specific user input. Some examples of user inputs that could be monitored for by device 10 are:
Where display 14 is touch-sensitive, a particular sequence of one or more touches or taps on display 14.
Sliding a finger across display 14 (optionally in a specific direction).
Drawing a particular gesture on display 14 with a finger.
Drawing a circle or the like on display 14 (optionally in a specific sense—clockwise or counterclockwise).
Holding down one or more control buttons for a period.
etc.

In the illustrated embodiment of FIG. 6, device 10 remains in idle loop 61 for more than a threshold time period, then device 10 is placed in a standby mode 62 from which device 10 must be woken up before it can be used. Block 63 waits for user input of the type required to trigger device 10 to wake up. If no input is detected then device 10 remains in standby mode 62. Otherwise, device 10 returns to block 58 until it is again ready to image. If device 10 receives an instruction to proceed, device 10 reads its battery status in block 64 and, based upon the battery status read in block 64, selects and loads an appropriate imaging sequence in block 65.

Device 10 sets the imaging sequence to operate according to a currently appropriate power mode in block 66. In block 67, signal-processing subsystem 22 is configured according to the appropriate configuration data and proceeds to acquire ultrasound images in block 68. If device 10 receives a command to freeze or idle or if device 10 detects that it has not been used for some time then the transmit receive circuitry is shut down in block 69 and device 10 returns to idle loop 61.

Device 10 may include an alarm (e.g. an audible or visible alarm) or other user interface component that alerts a user to upcoming changes in power mode and/or alerts the user prior to the battery becoming exhausted to the point that it can no longer maintain operation of device 10.

In some embodiments, device 10 is configured to save its current settings prior to the battery becoming exhausted. For example, device 10 could be configured to automatically save information identifying its current operational status in the event that the available battery power falls below some threshold (for example 5% charge). In devices which save such settings information:
device 10 could be configured to automatically resume operation according to the saved settings; or
device 10 may present the user with an option to resume operation using the saved settings;
   upon the battery being replaced or recharged or upon an alternative source of power becoming available.

In some embodiments, where device 10 is intended to be used in a sterile environment, device 10 may be enclosed in a sterile cover. In such environments it may not be convenient to change batteries of device 10 when those batteries become low. In some embodiments, device 10 includes a pickup coil that can receive electromagnetic energy from another coil, such as the primary of a transformer, and a charger that applies received electromagnetic energy to recharge battery 30. In such embodiments, device 10 may be placed adjacent to the other pickup coil so that it can receive enough electrical power by way of alternating electromagnetic fields transmitted through the sterile cover to recharge battery 30 and/or maintain the operation of device 10 when battery 30 has become depleted.

In some embodiments, software running on processor 20 or circuitry which is included in signal-processing subsystem 22 determines when the transducer assembly 16 is not contacting a surface (e.g. is in free air). Under such circumstances, device 10 may cease performing imaging or other ultrasound operations to save power. When in this mode, device 10 may check periodically to determine whether surface contact has been reestablished. For example, device 10 may periodically cause ultrasound to be emitted by transducer assembly 16 and check for reflected ultrasound signals that are indicative of transducer assembly 16 being against a surface of a subject (this may be done, for example, a few times every second).

Device 10 may include any of various mechanisms to monitor a state of charge of battery 30. By way of example, device 10 may comprise:
a circuit for monitoring an output voltage of battery 30;
a timer for indicating a cumulative use time of battery 30 since charged;
an integrator that integrates current delivered by battery 30;
some combination thereof; or
the like.

Certain implementations of the invention comprise computer processors which execute software instructions which cause the processors to perform a method of the invention. For example, one or more processors in an ultrasound device may implement power management methods as described herein by executing software instructions in a program memory accessible to the processor(s). The invention may also be provided in the form of a program product. The program product may comprise any medium which carries a set of computer-readable signals comprising instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

While a number of exemplary aspects and embodiments have been discussed above, those of skill in the art will recognize certain modifications, permutations, additions and sub-combinations thereof. For example, the invention may be applied to conserve electrical power in ultrasound devices that are not portable and/or ultrasound devices not powered by batteries.

What is claimed is:

1. A method for operating a portable ultrasound device comprising: a power supply; a transducer assembly; an ultrasound transmitter configured to drive the transducer assembly to emit ultrasound; and, an ultrasound receiver configured to detect ultrasound received at the transducer assembly;
the method comprising:
operating the portable ultrasound device in a power-saving idle mode;
while operating the portable ultrasound device in the idle mode, periodically driving the ultrasound transducer assembly to emit ultrasound and determining whether the ultrasound receiver has received reflected ultrasound indicative of the transducer assembly being against a surface of a subject; and,
in response to determining that the ultrasound receiver has received reflected ultrasound indicative of the transducer assembly being against a surface of a subject, automatically switching the ultrasound device to operate in a first power mode.

2. A method according to claim 1 comprising, while operating the ultrasound device in the first power mode, determining that the power supply has less than a threshold amount of available power;
in response to determining that the power supply has less than the threshold amount of available power, switching the ultrasound device from the first power mode to a second power mode; and
operating the ultrasound device in the second power mode;
wherein, in the first power mode, the ultrasound device consumes electrical power from the power supply at a rate greater than in the second power mode.

3. A method according to claim 2 wherein operating the ultrasound device in the second power mode comprises operating the ultrasound device to acquire ultrasound image data at a frame rate that is less than a frame rate at which ultrasound image data is acquired in the first power mode.

4. A method according to claim 2 wherein operating the ultrasound device in the second power mode comprises operating the ultrasound device to acquire ultrasound image data at a line density that is reduced relative to a line density provided in the first power mode.

5. A method according to claim 2 wherein operating the ultrasound device in the second power mode comprises transmitting ultrasound signals at a power that is reduced in comparison to a power of ultrasound signals transmitted by the ultrasound device while operating in the first power mode.

6. A method according to claim 2 wherein operating the ultrasound device in the first and second power modes comprises acquiring and processing frames of ultrasound image data wherein operating the ultrasound device in the second power mode comprises processing fewer than all frames of ultrasound image data that are acquired.

7. A method according to claim 6 wherein operating the ultrasound device in the second power mode comprises processing every Nth frame of acquired ultrasound data, where N is an integer.

8. A method according to claim 2 wherein switching from the first power mode to the second power mode comprises reconfiguring a configurable signal processing circuit.

9. A method according to claim 2 wherein the transducer assembly comprises a plurality of transducer elements and, in the second power mode, the ultrasound transmitter drives fewer of the transducer elements of the transducer assembly than the first power mode.

10. A method according to claim 2 wherein the transducer assembly comprises a plurality of transducer elements and, in the second power mode, the ultrasound device processes data from fewer transducer elements of transducer assembly than in the first power mode.

11. A method according to claim 2 wherein operating in the first and second power modes respectively comprise processing ultrasound data in first and second numbers of channels wherein the first number of channels is larger than the second number of channels.

12. An ultrasound device comprising:
a transducer assembly comprising a plurality of ultrasound transducer elements;
an ultrasound transmitter configured to drive the ultrasound transducer assembly to emit ultrasound;
an ultrasound receiver configured to detect ultrasound received at the transducer assembly;
a power supply;
a power supply monitoring system; and
a power management controller having an idle mode wherein the power management controller is configured to cause the transmitter to periodically drive the ultrasound transducer assembly to emit ultrasound and to determine whether the ultrasound receiver has received reflected ultrasound indicative of the transducer assembly being against a surface of a subject and, in response to the reception of reflected ultrasound indicative of the transducer assembly being against a surface of a subject switching to an operating mode.

13. An ultrasound device according to claim 12 wherein the power supply monitoring system is configured to provide an output indicative of available power from the power supply, wherein the power management controller is configured to monitor the output of the power management system and, in response to the output indicating that the power supply has less than a threshold amount of available power, switch the ultrasound device from a first power mode of the operating mode to a second power mode of the operating mode and operate the ultrasound device in the second power mode and wherein, in the first power mode, the ultrasound device consumes electrical power from the power supply at a rate greater than in the second power mode.

14. An ultrasound device according to claim 13 wherein the operating mode is one of a plurality of operating modes and, for each of the operating modes, the ultrasound device has a plurality of power modes.

15. An ultrasound device according to claim 14 wherein the ultrasound device comprises a field programmable gate array and a data store containing configuration information for the field programmable gate array for each of a plurality of the power modes for one of the operating modes wherein the power management controller is configured to reconfigure the field programmable gate array according to the configuration information corresponding to a power mode to which the power management controller is switching the ultrasound device.

16. An ultrasound device according to claim 14 comprising a data store specifying values for a plurality of parameters that affect the operation of the ultrasound device for each one of the power modes.

17. An ultrasound device according to claim 14 wherein the plurality of operating modes includes a B-mode imaging mode.

18. An ultrasound device according to claim 12 comprising a user control operable to selectively decrease ultrasound imaging performance and increase operating time.

19. An ultrasound device according to claim 18 comprising a display wherein the power controller is configured to estimate available operating time in a current power mode and display the estimate on the display.

20. A method for operating a portable ultrasound device comprising: a power supply; a transducer assembly; an ultrasound transmitter configured to drive the transducer assembly to emit ultrasound; and, an ultrasound receiver configured to detect ultrasound received at the transducer assembly;
the method comprising:
operating the device in a first power mode;
while operating the device in the first power mode, determining that the power supply has less than a threshold amount of available power;
in response to determining that the power supply has less than the threshold amount of available power switching the ultrasound device from the first power mode to a second power mode and operating the ultrasound device in the second power mode;
wherein operating the ultrasound device in the second power mode comprises any one or more of:
operating the ultrasound device to acquire ultrasound image data at a frame rate that is less than a frame rate at which ultrasound image data is acquired in the first power mode;
operating the ultrasound device to acquire ultrasound image data at a line density that is reduced relative to a line density provided in the first power mode;
transmitting ultrasound signals at a power that is reduced in comparison to a power of ultrasound signals transmitted by the ultrasound device while operating in the first power mode;
processing fewer than all frames of ultrasound image data that are acquired;
driving fewer transducer elements within the transducer assembly than a number of transducer elements within the transducer assembly that are driven in the first power mode;
processing data from fewer transducer elements within the transducer assembly than a number of transducer elements within the transducer assembly that are processed in the first power mode; and
processing ultrasound data in fewer channels than a number of channels processed in the first power mode.

21. A method according to claim 20 wherein operating the ultrasound device in the second power mode comprises any two or more of:
operating the ultrasound device to acquire ultrasound image data at a frame rate that is less than a frame rate at which ultrasound image data is acquired in the first power mode;
operating the ultrasound device to acquire ultrasound image data at a line density that is reduced relative to a line density provided in the first power mode;
transmitting ultrasound signals at a power that is reduced in comparison to a power of ultrasound signals transmitted by the ultrasound device while operating in the first power mode;
processing fewer than all frames of ultrasound image data that are acquired;
driving fewer transducer elements within the transducer assembly than a number of transducer elements within the transducer assembly that are driven in the first power mode;
processing data from fewer transducer elements within the transducer assembly than a number of transducer elements within the transducer assembly that are processed in the first power mode; and
processing ultrasound data in fewer channels than a number of channels processed in the first power mode.

22. A method according to claim 20 comprising:
operating the portable ultrasound device in a power-saving idle mode;
while operating the portable ultrasound device in the idle mode, periodically driving the ultrasound transducer assembly to emit ultrasound and determining whether the ultrasound receiver has received reflected ultrasound indicative of the transducer assembly being against a surface of a subject; and,
in response to determining that the ultrasound receiver has received reflected ultrasound indicative of the transducer assembly being against a surface of a subject, automatically switching the ultrasound device to operate:
in the first power mode when the power supply is determined to have more than the threshold amount of available power; or
in the second power mode when the power supply is determined to have more less the threshold amount of available power.

23. A method according to claim 20 comprising:
operating the device in the second power mode;
while operating the device in the second power mode, determining that the power supply has less than a lower threshold amount of available power;
in response to determining that the power supply has less than the lower threshold amount of available power switching the ultrasound device from the second power mode to a third power mode and operating the ultrasound device in the third power mode;
wherein operating the ultrasound device in the third power mode comprises any one or more of:
operating the ultrasound device to acquire ultrasound image data at a frame rate that is less than a frame rate at which ultrasound image data is acquired in the second power mode;
operating the ultrasound device to acquire ultrasound image data at a line density that is reduced relative to a line density provided in the second power mode;
transmitting ultrasound signals at a power that is reduced in comparison to a power of ultrasound signals transmitted by the ultrasound device while operating in the second power mode;
processing fewer than all frames of ultrasound image data that are acquired and fewer than a number of frames that are processed while operating in the second power mode;
driving fewer transducer elements within the transducer assembly than a number of transducer elements within the transducer assembly that are driven in the second power mode;
processing data from fewer transducer elements within the transducer assembly than a number of transducer elements within the transducer assembly that are processed in the second power mode; and
processing ultrasound data in fewer channels than a number of channels processed in the second power mode.

24. An ultrasound device comprising:
a transducer assembly comprising a plurality of ultrasound transducer elements;
an ultrasound transmitter configured to drive the ultrasound transducer assembly to emit ultrasound;
an ultrasound receiver configured to detect ultrasound received at the transducer assembly;
a power supply;
a power supply monitoring system configured to provide an output indicative of available power from the power supply; and
a power management controller configured to monitor the output of the power management system and, in response to the output indicating that the power supply has less than a threshold amount of available power, switch the ultrasound device from a first power mode of the operating mode to a second power mode of the operating mode and operate the ultrasound device in the second power mode;
wherein, in the first power mode, the ultrasound device consumes electrical power from the power supply at a rate greater than in the second power mode.

25. An ultrasound device according to claim 24 wherein the operating mode is one of a plurality of operating modes and, for each of the operating modes, the ultrasound device has a plurality of power modes.
